(19) United States
(12) Patent Application Publication
(10) Pub. No.: US 2007/0135827 A1
(43) Pub. Date: Jun. 14, 2007

(54) Method and apparatus to penetrate soft tissue and produce passageway in hard tissue while protecting principal vasculature and nerves

(76) Inventor: Richard I. Zipnick, Scottsdale, AZ (US)

Correspondence Address: TOD R NISSLE, PO BOX 55630, PHOENIX, AZ, 85078, US

(21) Appl. No.: 11/299,395

(22) Filed: Dec. 12, 2005

(51) Int. Cl. A61B 17/32

(52) U.S. Cl. 606/171

(57) ABSTRACT

A method and apparatus are provided to form an opening in tissue. The apparatus oscillates a distal end through soft and hard tissue at varying desirable frequencies and amplitudes. The method also facilitates detection of principal nerves and vasculature to minimize the risk of injury to the same.

This invention pertains to a method and apparatus for minimizing injury while penetrating tissue.

More particularly, this invention pertains to an apparatus and method for simplifying existing procedures for penetrating an intervertebral disc and other hard tissue.

An intervertebral disc interconnects vertebra bones in a spinal column. The disc includes an annulus and a nucleus. As used herein, the annulus is a hard tissue compartment that houses soft tissue comprising the nucleus. Other hard tissue found in the body includes bone, cartilage, and the capsules located at the end of bones at the joints of the hand, wrist, elbow, shoulder, foot, ankle, knee, and hip. Soft tissue in the body includes epithelium, fascia, muscle, fat, vasculature, and nerves.

Vasculature and nerves of differing width, or diameter, exist throughout the body. The larger vasculature and nerves are deemed principal vasculature and nerves. The lesser vasculature and nerves are deemed minor vasculature and nerves. As used herein, principal vasculature and nerves have a width of at least one millimeter (mm).

An object of many surgical procedures is to produce an opening in an intervertebral disc or other hard tissue including cartilage, bone, and the capsules around joints. During these surgical procedures, the distal end of an instrument often is passed through soft tissue in order to reach the hard tissue in which the opening is to be formed. Since the distal end of the instrument often has a sharp tip or cutting edge that is used to form an opening in the hard tissue, there is a significant risk that the distal end will cut or pierce principal vasculature or nerves and produce a serious injury, possibly a life threatening injury.

Accordingly, it would be highly desirable to provide an improved method and apparatus for forming an opening in hard tissue.

Therefore, it is a principal object of the instant invention to provide an improved method and apparatus for penetrating hard and soft tissue while minimizing the risk of injury to the tissue.

These and other, further and more specific objects and advantages of the invention will be apparent from the following detailed description of the invention, taken in conjunction with the drawings, in which:

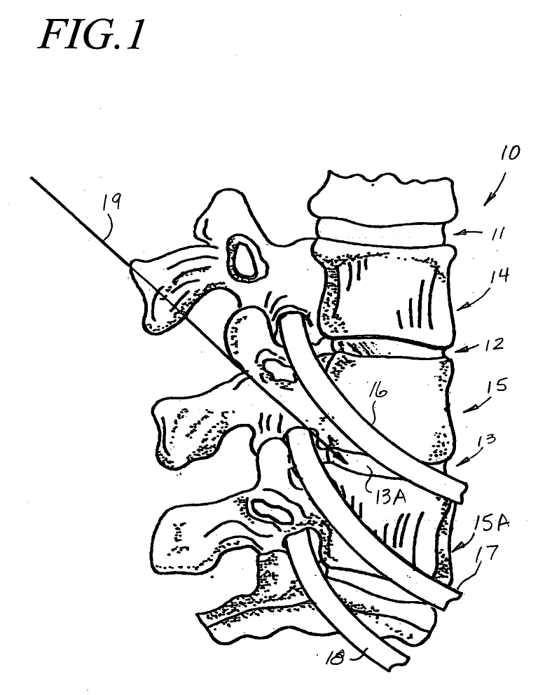

FIG. 1 is a side elevation view of a portion of a spine illustrating principal nerves that exit the spine;

FIG. 2 is a side view illustrating an instrument constructed in accordance with the principles of the invention to minimize the risk of injury to soft tissue and hard tissue while producing an opening in the hard tissue;

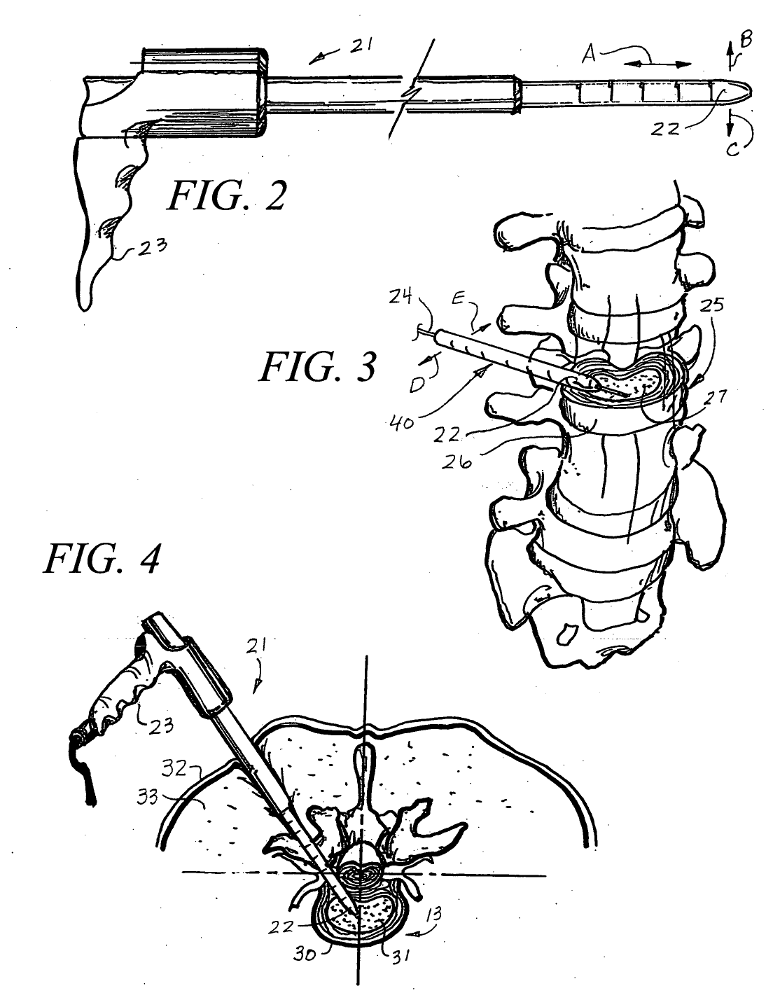

FIG. 3 is a front view of a portion of a spine illustrating the insertion along a wire of an instrument constructed in accordance with the invention;

FIG. 4 is a top view illustrating the mode of operation of the instrument of FIG. 2;

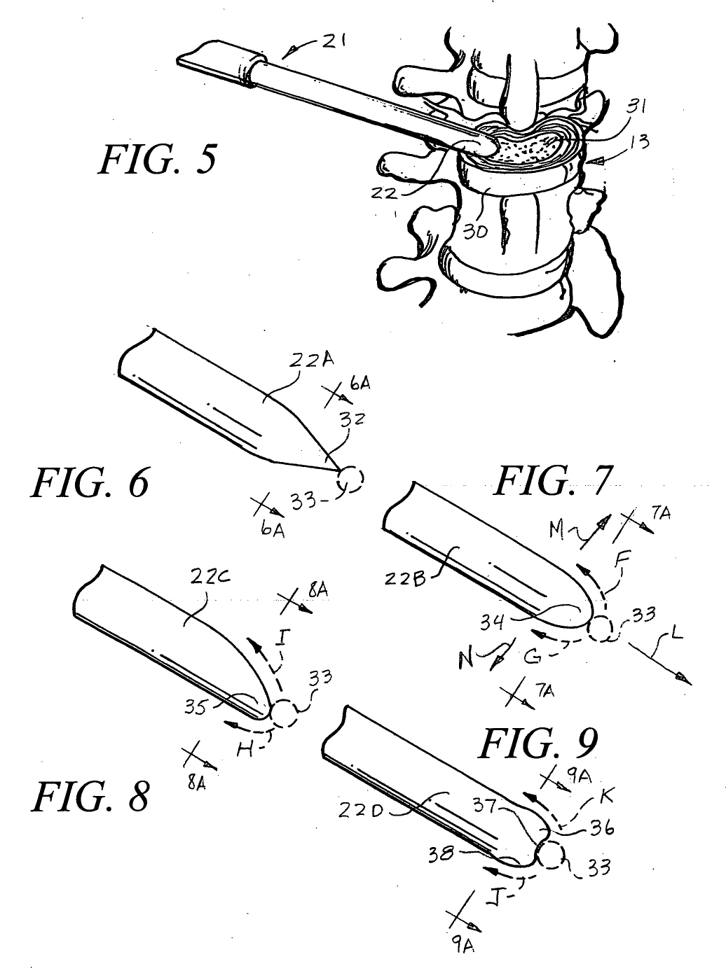

FIG. 5 is a front view further illustrating the mode of operation of the instrument of FIG. 2;

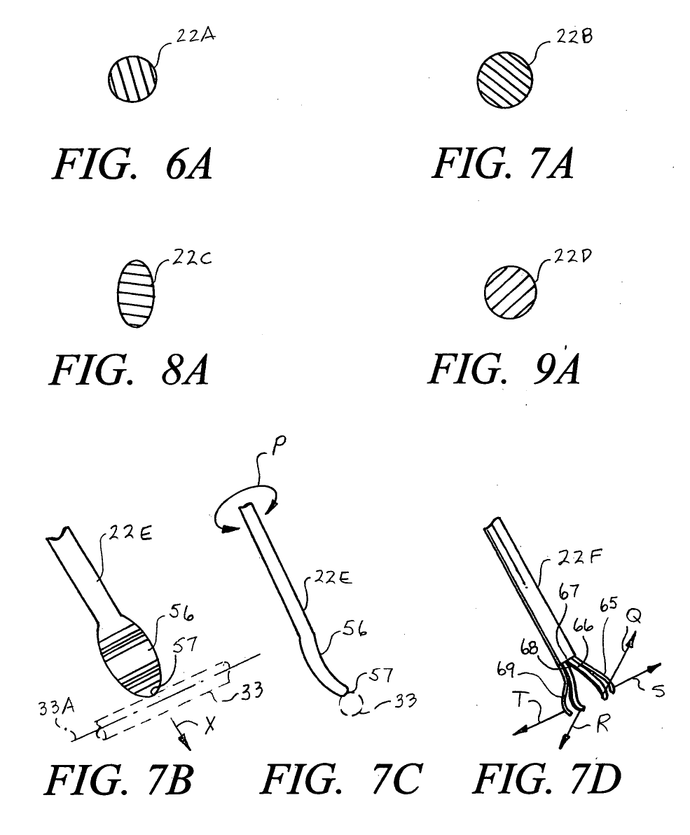

FIG. 6 is a top view illustrating an instrument construction that is to be avoided in the practice of the invention;

FIG. 6A is a section view illustrating the instrument of FIG. 6 and taken along section line 6A-6A;

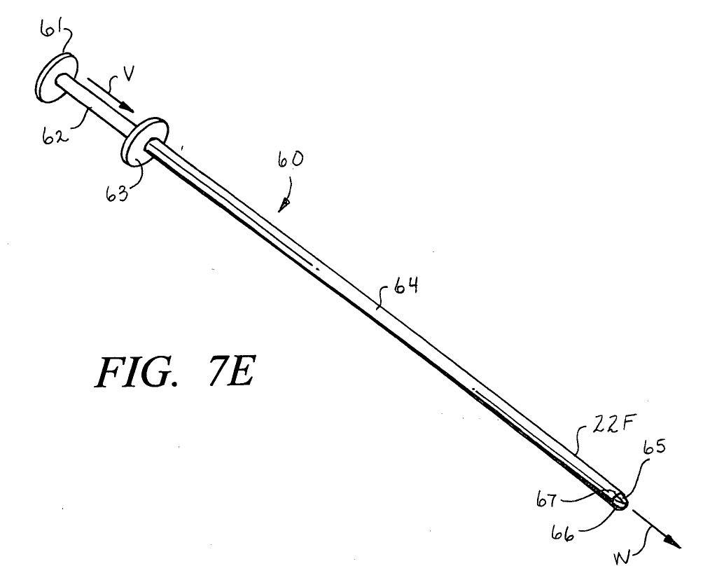

FIG. 7 is a top view illustrating an instrument construction that can be utilized in the practice of the invention;

FIG. 7A is a section view illustrating the instrument of FIG. 7 and taken along section line 7A-7A;

FIG. 7B is a top view illustrating another instrument constructed in accordance with the invention;

FIG. 7C is a side view illustrating the instrument of FIG. 7B;

FIG. 7D is a top view illustrating a further instrument constructed in accordance with the invention;

FIG. 7E is a perspective view illustrating the mode of operation of the instrument of FIG. 7D;

FIG. 8 is a top view illustrating another instrument construction that can be utilized in accordance with the invention;

FIG. 8A is a section view illustrating the instrument of FIG. 8 and taken along section line 8A-8A;

FIG. 9 is a top view illustrating a further instrument construction that can be utilized in the invention;

FIG. 9A is a section view illustrating the instrument of FIG. 9 and taken along section line 9A-9A;

FIG. 10 is a top view further illustrating the insertion of the instrument of FIG. 3 in an intervertebral disc along a wire;

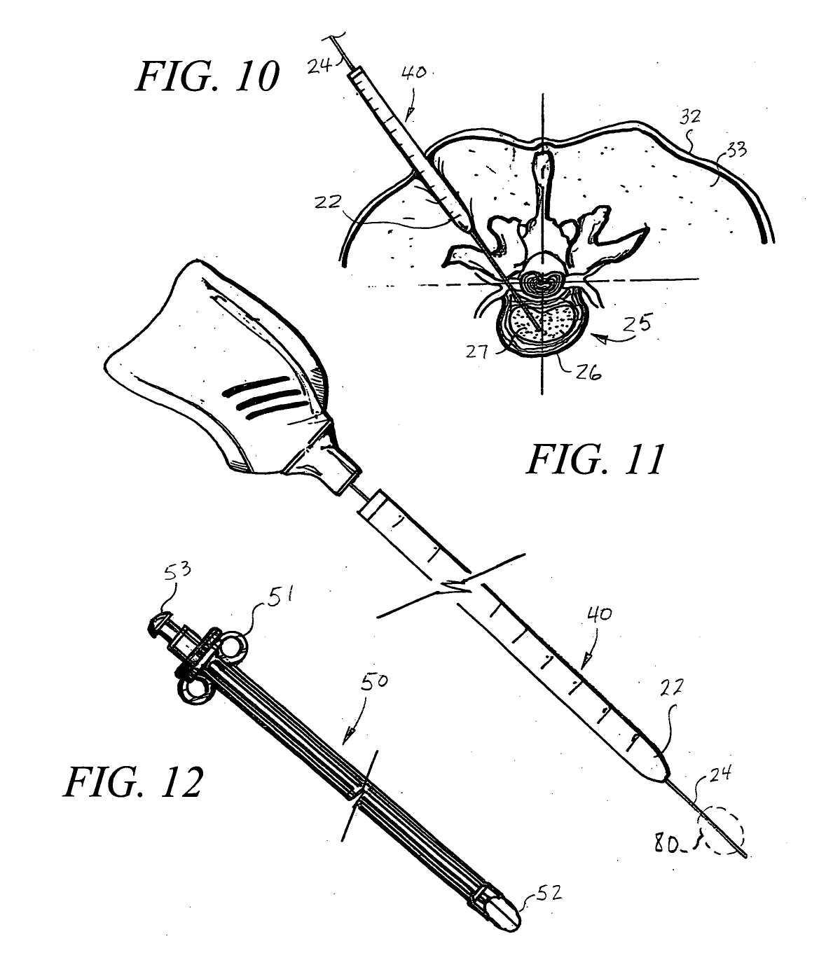

FIG. 11 is a side view further illustrating the instrument of FIG. 3;

FIG. 12 is a side view of an instrument that functions both to produce an opening in hard tissue and to insert an implant once the opening has been produced.

Briefly, in accordance with my invention, I provide an improved method to separate tissue. The improved method comprises the steps of providing an instrument shaped and dimensioned to oscillate within tissue around nerves and vasculature; and, oscillating the instrument within tissue around nerves and vasculature.

In another embodiment of the invention, I provide an improved method to form an opening in an intervertebral disc. The method comprises the steps of providing an instrument shaped and dimensioned to oscillate within the intervertebral disc; and, oscillating the instrument within an intervertebral disc.

In a further embodiment of the invention, I provide an improved method to widen an opening in an intervertebral disc. The method comprises the steps of providing an instrument shaped and dimensioned to oscillate within the intervertebral disc; and, oscillating the instrument within the intervertebral disc.

In still another embodiment of the invention, I provide an improved method for forming an opening in hard tissue while minimizing the risk of injury to principal vasculature and nerves. The method comprises the steps of providing an instrument with a distal end shaped and dimensioned to penetrate, when oscillated in and out, soft tissue; and, shaped and dimensioned, when contacting a principal vasculature or nerve, to prevent said distal end from cutting or piercing the principal vasculature or nerve, and to enable the distal end to move past the principal vasculature or nerve. The distal end moves past the principal vasculature or nerve by being oscillated in directions toward and away from the vessel, and by being laterally displaced. When the distal end contacts and is impeded by the principal vasculature or nerve, a resistance to movement of the distal end is generated that, along with the location of the distal end, indicates that the distal end has contacted the principal vasculature or nerve. The method also comprises the steps of oscillating the distal end to pass through the soft tissue; of, when contacting the principal vasculature or nerve, laterally displacing and oscillating the distal end to move the distal end past the principal vasculature or nerve; and, of contacting the hard tissue and oscillating the distal end against the hard tissue to form an opening therein.

In still a further embodiment of the invention, I provide an improved method for forming an opening in hard tissue. The method comprises the steps of providing an instrument with a distal end shaped and dimensioned to penetrate, when oscillated in and out, soft tissue and hard tissue; of oscillating the distal end to pass through the soft tissue to contact the hard tissue; and, of oscillating the distal end against the hard tissue to form an opening therein.

In yet another embodiment of the invention, I provide an improved method for detecting principal vasculature and nerves. The improved method comprises the steps of providing an instrument with a distal end. The distal end is shaped and dimensioned to penetrate, when oscillated in and out, soft tissue; and, when contacting a principal circulatory/neural vessel, to prevent the distal end from cutting or piercing the principle circulatory/neural vessel. When the distal end contacts and is impeded by a principal vasculature or nerve, a resistance is generated that indicates that the distal end has contacted a principal circulatory/neural vessel. The method also comprises the step of oscillating the distal end to pass through the soft tissue until the resistance indicates that the distal end is contacting a principle circulatory/neural vessel.

In yet a further embodiment of the invention, I provide improved apparatus for forming an opening in hard tissue. The apparatus comprises an instrument with a tissue contacting rounded distal end shaped and dimensioned to penetrate, when oscillated, hard tissue. The distal end can be shaped and dimensioned, when contacting a principal vasculature or nerve, to prevent the distal end from cutting or piercing the principal vasculature or nerve, and to enable the distal end to move past the principal vasculature or nerve.

In yet still another embodiment of the invention, I provide an improved method of passing an implant through tissue to an intervertebral disc location. The method comprises the steps of providing an elongate guide unit; providing an implant structure shaped and dimensioned to pass through tissue and move along the guide unit; and, moving the implant structure through tissue along the guide unit to the intervertebral disc location.

Turning now to the drawings, which depict the presently preferred embodiments of the invention for the purpose of illustrating the practice thereof and not by way of limitation of the scope of the invention, and in which like reference characters refer to corresponding elements throughout the several views, FIG. 1 illustrates a portion 10 of a spinal column, including vertebrae 14, 15, 15A, and intervertebral discs 11, 12, 13. Principal nerves 16, 17, 18 emerge from the spinal column. Arrow 19 illustrates a preferred path for an instrument to travel in order to avoid nerves 16 and 17 and to impinge on the annulus 1 3A of disc 13. Path 19 may not, however, avoid impingement on a nerve 16, 17 in the event a nerve 16 happens to be in an unusual position, in the event disc 13 is squeezed into an bulging configuration that causes vertebrae 15 and 15A and nerves 16 and 17 to move closer together, etc.

FIGS. 2, 4, 5 illustrate apparatus 21 constructed in accordance with the invention and including a distal end 22 and handle 23. During insertion in the body of a patient, apparatus 21 is manually or mechanically oscillated back and forth in the direction of arrows A, oscillated up and down in the direction of arrows B and C, oscillated laterally in the direction of arrows E and D (FIG. 3), oscillated in a manner that combines movement in two or more of said directions A to E, i.e.,the distal end 22 can be moved along an elliptical or circular path, oscillated radially in and out in the manner of fingers 65, 66, 68, and 69 in FIG. 7D, and/or oscillated rotationally about the longitudinal axis of the apparatus in the manner indicated by arrows P in FIG. 7C. Since the purpose of moving end 22 is to produce an opening in and through tissue, the in-and-out oscillating movement indicated by arrows A (FIG. 2) is preferred and typically is required even if oscillating movement of end 22 in the direction of arrows B and C, in the direction of arrows E and D (FIG. 3), along a circular path, radially, or rotationally is also employed. The frequency and amplitude of oscillation can vary as desired, as can the force or pressure applied to handle 23 to press end 22 into tissue 32, 33 toward selected hard tissue 30 (FIG. 4). When passing end 22 through soft tissue, particularly soft tissue where there is no principal vasculature or nerves. A longer amplitude and smaller frequency is typically employed. When passing end 22 through hard tissue, a higher frequency and smaller amplitude typically is preferred. By way of example, and not limitation, the frequency of radial, linear, or rotational oscillation through soft tissue or hard tissue is greater than or equal to 0.1 cycles per minute. The amplitude of oscillation can vary as desired, but the amplitude of oscillation typically is greater in soft tissue than it is in hard tissue.

Apart from forward movement of a distal end 22, 22B to 22E (FIGS. 7, 8, 9, 7B, 7C) caused by oscillation, forward movement of a distal end 22 through soft tissue in a direction L (FIG. 7) can vary as desired, but typically is greater in soft tissue than it is in hard tissue.

The pressure required for a rounded distal end 22, 22B to 22E to tear or pierce or otherwise injure a principal nerve or vasculature varies depending on the shape of the tip of the end 22, 22B to 22E and on the size and makeup of the nerve or vasculature, but is readily determined by experimentation so that a surgeon can avoid applying pressure in the direction of travel L (FIG. 7), having a magnitude sufficient to injure a principal nerve or vasculature.

FIG. 4 illustrates the location of instrument 21 and distal end 22 after end 22 has been oscillated to pass through epithelium 32, through other soft tissue including fat, facia, muscle, minor vasculature and nerves, and principal vasculature and nerves, and through the annulus 30 of disc 13 into the nucleus 31. Since the epithelium 32 can be difficult to penetrate initially, a small incision can be made in epithelium 32 to facilitate the passage of end 22 therethrough.

The shape of end 22 is important. Various shapes of end 22 are illustrated in FIGS. 6 to 9, and in FIGS. 7B, 7C, 7D and 7E.

The distal end 22A in FIG. 6 has a sharp tip, or point, 32. Distal end 22A is not utilized in the practice of the invention because tip 32 can readily puncture or cut a principal nerve 33 or vasculature. Similarly, a distal end that includes a cutting edge is not preferred in the practice of the invention.

The distal end 22B illustrated in FIG. 7 has a rounded tip 34 and is a preferred construct in the practice of the invention. If tip 34 contacts a principal nerve 33 while moving and/or oscillating in the direction of arrow L, it is likely that nerve 33 will slide off to one of the sides indicated by arrows F and G. If, on the other hand, tip 34 contacts nerve 33 “dead on” and nerve 33 impedes the progress of tip 34 in the direction of arrow L, the surgeon that is manually oscillating instrument 21 will feel the resistance (or a sensor on a machine that is oscillating instrument 21 will detect the resistance) and can laterally displace tip 34 in the direction of arrow N or M to facilitate the movement of nerve 33 in the direction of arrow G or F over end 34 so that tip 34 can continue moving in the direction of arrow L. The surgeon increases the certainty that tip 34 has contacted principal nerve 33 or principal vasculature by determining the location of tip 34 with a fluoroscope, with an endoscope, by direct visualization, by patient feed back, by an electrical recording of a nerve, by an alteration of blood pressure or pulse rate caused by contacting a blood vessel, or any other desired means.

The distal end 22C illustrated in FIG. 8 has a rounded tip 35 and is also a preferred construct in the practice of the invention. If tip 35 contacts a principal nerve 33 or vasculature while moving and/or oscillating in a direction toward nerve 33, it is likely that nerve 33 will slide off to one of the sides of end 22C indicated by arrows H and 1. If, on the other hand, tip 35 contacts nerve 33 “dead on” and nerve 33 impedes the progress of tip 35, the surgeon that is manually oscillating instrument 21 (or a sensor on a machine that is oscillating instrument 21) will detect the resistance and can manipulate the handle 23 of instrument 21 (FIG. 4) to laterally displace tip 35 to facilitate the movement of nerve 33 in the direction of arrow H or I over end 35 so that tip 35 can continue moving past nerve 33. The surgeon increases the certainty that tip 35 has contacted principal nerve 33 or principal vasculature by determining the location in the patient's body of tip 35 with a fluoroscope, with an endoscope, by direct visualization, by patient feed back, by an electrical recording of a nerve, by an alteration of blood pressure or pulse rate caused by contacting a blood vessel, or any other desired means. Once the surgeon determines the location of tip 35, the surgeon's knowledge of the normal anatomy of an individual and/or knowledge of the patient's particular anatomy assists the surgeon in determining if a principal nerve or vasculature has been contacted by tip 35.

The distal end 22D illustrated in FIG. 9 has a rounded tips 36, 38 and detent 37 and is also a preferred construct in the practice of the invention. If tip 36 or 38 contacts a principal nerve 33 while moving and/or oscillating in a direction toward nerve 33, it is likely that nerve 33 will slide off to one of the sides of end 22D in a direction indicated by arrow K or J. If, on the other hand, detent 37 contacts nerve 33 “dead on” and nerve 33 seats in detent 37 and impedes the progress of end 22D, the surgeon that is manually oscillating instrument 21 will feel the resistance (or a sensor on a machine that is oscillating instrument 21 will detect the resistance) and can manipulate the handle 23 of instrument 21 (FIG. 4) to laterally displace distal end 22D to facilitate the movement of nerve 33 in the direction of arrow J or K over end 22D so that end 22D can continue moving past nerve 33. The surgeon increases the certainty that end 22D has contacted principal nerve 33 or principal vasculature by determining the location in the patient's body of tips 36, 38 with a fluoroscope, with an endoscope, by direct visualization by patient feed back, by an electrical recording of a nerve, by an alteration of blood pressure or pulse rate caused by contacting a blood vessel, or any other desired means. Once the surgeon determines the location of tips 36, 38, the surgeon's knowledge of the normal anatomy of a the body of a human being or animal and/or knowledge of the patient's particular anatomy, assists the surgeon in determining if a principal nerve or vasculature has been contacted by end 22D.

The spoon-shaped distal end 22E illustrated in FIG. 7B has a curved paddle surface 56 and a rounded edge 57 and is also a preferred construct in the practice of the invention. If rounded edge 57 contacts a principal nerve 33 while moving and/or oscillating in a direction toward nerve 33, it is likely that nerve 33 will slide off to one of the sides of end 22E. It is preferred that edge 57 contact nerve 33 (or principal vasculature) in the manner illustrated in FIG. 7B with surface 56 generally parallel to the longitudinal axis 33A of the nerve. If, on the other hand, edge 57 contacts nerve 33 in an orientation in which the spoon surface 56 of FIG. 7B is rotated ninety degrees such that surface 56 is generally normal to axis 33A, there is a greater risk of injury to nerve 33A. If edge 57 contacts nerve 33 “dead on” such that nerve 33 impedes the progress of end 22E in the direction of arrow X, the surgeon that is manually oscillating instrument 21 will feel the resistance (or a sensor on a machine that is oscillating instrument 21 will detect the resistance) and can manipulate the handle 23 of instrument 21 (FIG. 4) to laterally displace distal end 22E to facilitate the movement of nerve 33 laterally from edge 57 so that end 22E can continue moving past nerve 33. The surgeon increases his certainty that edge 57 has contacted principal nerve 33 or principal vasculature by determining the location in the patient's body of edge 57 with a fluoroscope, with an endoscope, by direct visualization, by patient feed back, by an electrical recording of a nerve, by an alteration of blood pressure or pulse rate caused by contacting a blood vessel, or any other desired means. Once the surgeon determines the location of edge 57, the surgeon's knowledge of the normal anatomy of a the body of a human being or animal and/or knowledge of the patient's particular anatomy assists the surgeon in determining if a principal nerve or vasculature has been contacted by end 22E.

The distal end 22F illustrated in FIG. 7D includes a plurality of curved fingers 65, 66, 68, and 69 depicted in their deployed, open position. The fingers are shown in FIG. 7E in their normal stowed position adjacent and in opening 67 formed in distal end 22F of instrument 60. In the stowed position, a substantial portion of fingers 65, 66, 68, and 69 is drawn through opening 67 to a position inside hollow cylindrical body 64. In the stowed position, however, the curved distal ends of fingers 65, 66, 68, and 69 extend outwardly from opening 67 in the manner illustrated in FIG. 7D and generally collectively form an arcuate surface similar to the surface on the end of an egg. Moving end 61 in the direction of arrow V causes neck 62 to slide into hollow cylindrical body 64 to displace fingers 65, 66, 68, and 69 outwardly in the direction of arrow W. When fingers 65, 66, 68, and 69 are outwardly displaced in the direction of arrow W, they open radially in the directions indicated by arrows Q, S, R, and T, respectively, to the expanded deployed position illustrated in FIG. 7D When end 61 is released, it moves in a direction opposite that of arrow V and returns to the position illustrated in FIG. 7E, and, similarly, fingers 65, 66, 68, and 69 move back to the stowed position illustrated in FIG. 7E. Consequently, repeatedly manually (or mechanically) pressing end 61 in the direction of arrow V and then releasing end 61 causes fingers 65, 66, 68, and 69 to oscillate radially in and out in the directions indicated by arrows Q to T, and causes fingers 65, 66, 68, and 69 to oscillate back and forth in the direction of arrow W and in a direction opposite that of arrow W. Rotating distal end 22E in FIG. 7C back and forth in the directions indicated by arrows P causes end 22E to oscillate back and forth. Continuously rotating end 22E also, practically speaking, causes end 22E to oscillate because of the flat spoon shape of end 22E.

FIG. 10 further illustrates the insertion of instrument 40 along wire 24 through epithelium 32 and other soft tissue 33 toward the annulus of disc 25.

FIG. 11 also illustrates instrument 40 slidably mounted on wire 24.

FIG. 12 illustrates an instrument 50 that is utilized to insert an implant 52 in the nucleus 27 of an intervertebral disc 26 (FIG. 3) or to insert the implant 52 in another location in a body. The rounded tip of the implant 52 functions in a manner equivalent to the rounded tips of distal ends 22B (FIG. 7), 22C (FIG. 8), 22D (FIG. 9), 22E (FIGS. 7B and 7C), and 22F (FIG. 7D) to facilitate the passage through tissue of the tip of implant 52. An implant 80 (FIG. 11) can have a rounded tip like implant 52, can function in a manner equivalent to the rounded tips of distal ends 22B, 22C, etc., and can also have an opening formed therethrough that permits implant 80 to slide or otherwise move along a wire 24 or other elongate member. The shape and dimension of the opening formed through implant 80 can vary as desired, as can the shape and dimension of the elongate member. If an opening of sufficient size exists in tissue and if wire 24 is appropriately oriented, implant 80 may slide along wire 24 of its own accord under the force of gravity to a desired location in a patient's body. Or, a surgeon's hand or hands or an auxiliary instrument 50 can be utilized to contact and move implant 80 along wire 24 to a desired location. As utilized herein, a distal end 22B, 22C, 22D, etc. can comprise an instrument that oscillates or otherwise moves through tissue, as can an implant 80. The combination of an auxiliary instrument 50 (FIG. 12) with a distal end 22B, 22C, 22D, etc. or implant 80 can also comprise an instrument as long as the combination functions in accordance with at least one of the principles of the invention and separates tissue, forms an opening in tissue, passes through tissue, and/or delivers an implant to a selected location in a patient's body. Grasping handle 51 and depressing member 53 releases implant 52 from instrument 50.

Forming an opening in tissue with a distal end 22 (FIG. 4) shaped and dimensioned in accordance with the invention requires the end 22 to produce radial forces that work to form an opening in tissue. The tapered configuration of the tips of distal ends 22, 22B to 22F facilitate the generation of such outwardly acting radial forces. The outward movement of fingers 65, 66, 68, 69 when moving from their stowed to their deployed position generates such radial forces. Rotating or oscillating distal end 22E (FIG. 7C) in the manner indicated by arrows P also generates such “opening widening” radial forces. An opening is formed either by widening an existing opening or by forming a opening in tissue at a location at which no opening previously existed.

Having described my invention in such terms as to enable those of skill in the art to make and practice it, and having described the presently preferred embodiments thereof, I claim:

1. A method of separating tissue, said method comprising the steps of
(a) providing an instrument shaped and dimensioned to oscillate within tissue around nerves and vasculature; and,
(b) oscillating said instrument within tissue around nerves and vasculature.

2. A method of forming an opening in an intervertebral disc comprising the steps of
(a) providing an instrument shaped and dimensioned to oscillate within the intervertebral disc; and,
(b) oscillating said instrument within the intervertebral disc.

3. A method of widening an opening in an intervertebral disc comprising the steps of
(a) providing an instrument shaped and dimensioned to oscillate within the intervertebral disc; and,
(b) oscillating said instrument within the intervertebral disc.

4. A method of passing an implant through tissue to an intervertebral disc location, comprising the steps of
(a) providing an elongate guide unit;
(b) providing an implant structure shaped and dimensioned to pass through tissue and move along said guide unit; and,
(c) moving said implant structure through tissue along said guide unit to the intervertebral disc location.
